(19) United States
(12) Patent Application Publication
(10) Pub. No.: US 2004/0241243 A1
(43) Pub. Date: Dec. 2, 2004

(54) Angiogenesis promotion by prostaglandin compositions and methods

(75) Inventors: Guiting Lin, San Francisco, CA (US); Y. Joseph Mo, Princeton, NJ (US); Mingqi Lu, Lawrenceville, NJ (US); Yinglu Guo, Beijing (CN)

Correspondence Address: BOWDITCH & DEWEY, LLP, 161 WORCESTER ROAD, P.O. BOX 9320, FRAMINGHAM, MA, 01701-9320, US

(73) Assignee: NexMed (Holdings) Inc., Robbinsville, NJ

(21) Appl. No.: 10/803,101

(22) Filed: Mar. 16, 2004

Related U.S. Application Data
(60) Provisional application No. 60/456,605, filed on Mar. 21, 2003.

(51) Int. Cl. A61K 31/557; A61K 9/14

(52) U.S. Cl. 424/486; 514/573

(57) ABSTRACT

The present invention provides compositions and methods for promoting the recovery of vascular function in erectile dysfunction associated with vasculopathy by administering a composition comprising a vasoactive prostaglandin and a biocompatible polymer. In preferred embodiments, the prostaglandin composition is a topical composition comprising prostaglandin E₁, a biocompatible polymer and a penetration enhancer and the topical composition is applied to the meatus at the tip of the penis. In another embodiment, the invention provides a method for increasing microvascular outgrowth at a targeted arterial segment comprising administering a prostaglandin E₁ composition to produce an extracellular prostaglandin E₁ concentration of about 1 micromolar to about 10 micromolar adjacent to the targeted arterial segment for about four days.

### CROSS REFERENCES TO RELATED APPLICATIONS

The present application claims the benefit of U.S. Provisional Application No. 60/456,605, filed Mar. 21, 2003, which is incorporated herein by reference in its entirety.

### BACKGROUND OF THE INVENTION

The arterial blood supply to the erectile tissue of the penis is provided by the cavernosal (deep penile) and dorsal (superficial penile) arteries, which are terminal branches of the internal pudendal artery. The internal pudendal artery generally arises from the anterior division of the hypogastric or internal iliac artery. Helicine arteries, coiled in the flaccid penis, are terminal branches of the deep and dorsal arteries of the penis. Parasympathetic stimulation causes the helicine arteries to uncoil, allowing blood at arterial pressure to fill the cavernous tissue, causing an erection. Normal erectile function requires functional arterial blood supply, as well as the proper function of the smooth muscle cells and endothelial cells of the penile vasculature and erectile tissue.

Diabetes mellitus is a common risk factor in erectile dysfunction (ED). However the pathogenesis of ED in diabetes is not completely understood (Sullivan, M. E., et al., Alterations in endothelin B receptor sites in cavernosal tissue of diabetic rabbits: potential relevance to the pathogenesis of erectile dysfunction. J Urol. 1997 158(5):1966-72). ED in diabetes may be one aspect of vascular disease associated with diabetes (Sairam, K., et al., Prevalence of undiagnosed diabetes mellitus in male erectile dysfunction. BJU Int., 2001, 88(1):68-71; Sullivan, M. E., et al. Nitric oxide and penile erection: is erectile dysfunction another manifestation of vascular disease? Cardiovasc Res., 1999, Aug 15, 43(3):658-65)

Microvasculopathy is one of the characteristics of diabetes. Studies have suggested a link between diabetes, erectile dysfunction and endothelial cells dysfunction (De Angelis, L., et al., Erectile and endothelial dysfunction in Type II diabetes: a possible link. Diabetologia, 2001, 44(9):1155-60; Burchardt, T., et al., Reduction of endothelial and smooth muscle density in the corpora cavernosa of the streptozotocin induced diabetic rat. J Urol. 2000 164(5): 1807-11; Hopfner, R. L., & Gopalakrishnan, V., Endothelin: emerging role in diabetic vascular complications. Diabetologia. 1999 42(12):1383-94).

The angiogenic activity of prostaglandin E₁ (PGE₁) and prostaglandin E₂ (PGE₂) has been reported using various in vitro systems (BenEzra, D., Neovasculogenic ability of prostaglandins, growth factors, and synthetic chemoattractants. Am J Ophthalmol., 1978, 86(4):455-61; Form, D. M., & Auerbach, R., PGE2 and angiogenesis. Proc Soc Exp Biol Med., 1983, 172(2):214-8). PGE₁ has been reported to be involved in angiogenesis in models on tumor vascularization (Ziche, M., et al., Role of prostaglandin E₁ and copper in angiogenesis. J Natl Cancer Inst., 1982, 69(2):475-82).

The administration of erection effecting and enhancing drugs is taught in U.S. Pat. No.4,127,118 to LaTorre. This patent teaches a method of treating male impotence by injecting into the penis an appropriate vasodilator, in particular, an adrenergic blocking agent or a smooth muscle relaxant to effect and enhance an erection.

More recently, U.S. Pat. No. 4,801,587 to Voss et al. teaches the application of an ointment to relieve impotence. The ointment consists of the vasodilators papaverine, hydralazine, sodium nitroprusside, phenoxybenzamine, or phentolamine and a carrier to assist absorption of the primary agent through the skin. U.S. Pat. No. 5,256,652 to E1-Rashidy teaches the use of an aqueous topical composition of a vasodilator such as papaverine together with hydroxypropyl-β-cyclodextrin.

Prostaglandin E₁ (PGE₁) is a derivative of prostanoic acid, a 20-carbon atom lipid acid, represented by the formula:

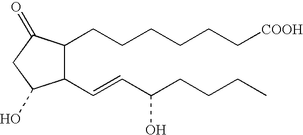

and is commercially available, e.g., from Chinoin Pharmaceutical and Chemical Works Ltd. (Budapest, Hungary) under the designation “Alprostadil USP,” from Pharmacia & Upjohn under the designation “Caverject”. Prostaglandin E₁ complexed with alpha-cyclodextrin is available as alprostatil alfadex from Ono Pharmaceuticals (Japan) and in an injectable form under the designation “Edex®” or “Viradex®” from Schwarz Pharma (Germany). Intracavernosal injection of prostaglandin E₁, alone or in combination with phentolamine and/or papavarine, remains a standard diagnostic and therapeutic for erectile dysfunction. However, scarring and pain at the injection site has reduced patient acceptance of intracavernosal injection as a routine or chronic treatment method.

In one commercially available form (MUSE®, Vivus, Menlo Park Calif.), alprostadil is administered transurethrally as a pellet deposited in the urethra using an applicator with a hollow stem 3.2 cm in length and 3.5 mm in diameter (Padma-Nathan, H., et al., N. Engl. J. Med., 1997, 336: 1-7, see especially FIG. 1). In the home treatment portion of the Padma-Nathan et al. study, 32.7% of the patients (10.8% of administrations) receiving MUSE® complained of penile pain and 5.1% experienced minor urethral trauma, compared to 3.3% and 1.0%, respectively, of the patients receiving placebo. Frequency of report of these side effects has varied in subsequent studies: MUSE® producing penile pain in 17-23.6% of administrations, compared to 1.7% with placebo and minor urethral bleeding reported by 4.8% of patients (Peterson, C. A., et al., J. Urol., 1998, 159: 1523-1528). In a study on a European population, 31% MUSE® patients reporting penile pain or burning sensations, 4.8% reporting urethral bleeding, and 2.9% reporting severe testicular pain (Porst, H., Int. J. Impot. Res., 1997, 9:187-192). The percent of patients responding to MUSE® treatment, defined as having at least one erection considered sufficient for intercourse, has been reported to be 43% (Porst, 1997), 65.9% (Padma-Nathan et al., 1997) and 70.5% (Peterson et al., 1998), although published editorial comment has suggested that the percent of patients responding in the latter two studies is more properly reported as 30-40% (Benson, G., J. Urol., 1998, 159: 1527-1528). Intraurethral application of a preparation of 1 mg prostaglandin E₁ in phosphatidylcholine liposomes in 1 ml polyoxyethylene glycol has been reported to be less effective than intracavernosal injection of prostaglandin E₁ (Englehardt, P. F., et al., British J. Urology, 1998, 81: 441-444).

Recently, intrameatal (or meatal) application of a topical PGE₁ composition comprising at least one penetration enhancer has been shown to be a non-invasive alternative to intracavernosal injection or transurethral suppositories for the treatment of erectile dysfinction (see U.S. Pat. No. 6,323,241, the contents of which are hereby incorporated in their entirety). Intrameatal application is the application of medication to the tip of the penis into the navicularfossa by holding the penis upright, holding the meatus open and dropping the medication into the navicularfossa, without introducing the medication container into the meatus.

One current hypothesis is that ischemic damage due to the hypoxic conditions under reduced oxygenation of the cavernosal tissue is a limiting factor in recovery of erectile function. The hypoxic conditions encourage the development of pathological fibrosis as well as the degeneration of cavernosal smooth muscle. It has been suggested that the oxygenated arterial blood flooding in during an erection would offset the effects of hypoxia. See, generally Novak, T. E., “Management of Erectile Dysfunction Following Radical Prostatectomy,” pp.109-122 in Mulcahy, J. J., ed., Male Sexual Function: A Guide to Clinical Management, Humana Press, Totowa, N.J., 2001.

There have been three brief reports of studies of the effects of intracavernous injections of prostaglandin E₁ after nerve-sparing retropubic radical prostatectomy. In one uncontrolled study, 31 of 40 patients completed the course of treatment (Padma-Nathan, H., et al., The impact on return of spontaneous erections of short-term Alprostadil therapy post nerve sparing prostatectomy, J. Urol., 1997, 157 (Suppl. 4): 363 (abstract 1422)). The subjects who began therapy less than 300 days after surgery had a more positive outcome than those who began therapy more than 300 days after surgery. In a prospective, randomized trial of intracavernous alprostadil injection after nerve sparing RRP, 12 of 15 patients completed the course of treatment (three months of intracavernous alprostadil injections three times a week), and 8 of the 12 reported a recovery of spontaneous erections sufficient for intercourse, compared to 3 of 15 untreated patients (Montorsi F, et al., The subsequent use of intracavernous alprostadil and oral sildenafil is more efficacious than sildenafil alone in nerve sparing radical prostatectomy patients, abstract presented at the 2002 annual meeting of the American Urology Association). The improvement attributed to improved cavernous oxygenation by the regime of alprostadil injection, limiting the development of hypoxia-induced tissue damage. A third study reported that not only were patients receiving three months of intracavernous alprostadil injections three times a week more likely to recover spontaneous erections, they were also more likely to be responsive to oral sildenafil therapy (Montorsi F., et al., 1997).

PGE₁ has been shown to produce an increase in intracellular levels of the second messenger cyclic adenosine monophosphate (cAMP) by binding to a specific membrane-bound receptor of the EP₂ or EP₄ subclasses (Narumiya, S., et al., Prostanoid receptors: Structures, Properties and Functions, Physiological Reviews, 1999, 79: 1193-1226). The affinity of either PGEI, or the endogenous ligand PGE₂, for the EP₂ receptor is reported to be about 10 nM and about 2 nM for the EP₄ receptor (Narumiya, S., et al., 1999). Activation of the EP₂ or EP₄ receptors by ligand binding relaxes smooth muscle (Zhang, Y., et al., Characterization of murine vasopressor and vasodepressor prostaglandin E₂ receptors, Hypertension, 2000, 35: 1129-1134).

The increase in cAMP levels is produced by the binding of PGE₁ or the endogenous ligand PGE₂, to a specific membrane bound receptor of the subclasses EP₂ or EP₄ (Narumiya, S., et al., 1999). The affinity of either PGE₁ or PGE₂ for the EP₂ receptor is about 10 nM and for the EP₄ receptor is about 2 nM (Narumiya, S., et al., 1999). Activation of the EP₂ or EP₄ receptors by ligand binding relaxes smooth muscle (Zhang, Y., et al., 2000). In the penile tissue PGE₁ activates cAMP production, thereby inducing smooth muscle relaxation and producing penile erection.

A study carried out in rats reported an improvement in neurogenic and vasculogenic erectile dysfunction associated with hypercholesterolemia by treatment with vascular endothelial growth factor (VEGF) and adeno-associated virus (AAV) mediated, brain derived neurotrophic factor (BDNF) (Gholami, S. S., et al., The effect of vascular endothelial growth factor and adeno-associated virus mediated brain derived neurotrophic factor on neurogenic and vasculogenic erectile dysfimction induced by hyperlipidemia. J Urol., 2003, 169(4):1577-1581). Prostaglandins can increase the production of VEGF. PGE₂ has been shown to up-regulate VEGF in vitro in endothelial cells (Pai, R., et al., PGE(2) stimulates VEGF expression in endothelial cells via ERK2/JNK1 signaling pathways. Biochem Biophys Res Commun., 2001, 286(5):923-8.). Treatment of patients with systemic PGE₁ has been reported to up-regulate expression of VEGF (Mehrabi, M. R., et al., Clinical and experimental evidence of prostaglandin E₁-induced angiogenesis in the myocardium of patients with ischemic heart disease, Cardiovasc Res., 2002, 56(2):214-24).

### SUMMARY OF THE INVENTION

The present invention provides compositions and methods for increasing microvascular sprouting from a targeted arterial segment using a composition including a vasoactive prostaglandin and a biocompatible polymer. In another aspect, the present invention provides compositions and methods for improving vascular fuiction in patients having erectile dysfunction associated with vasculopathy, such as diabetic vasculopathy.

In one embodiment, the present invention provides a convenient and non-invasive method of promoting the recovery of vascular function in erectile dysfunction associated with vasculopathy by meatally administering a composition comprising a vasoactive prostaglandin selected from the group consisting of prostaglandin E₁ (PGE₁), prostaglandin E₂ (PGE₂), pharmaceutically acceptable salts thereof, lower alkyl esters thereof, mixtures thereof and a biocompatible polymer thickener. In preferred embodiments, the composition is a topical composition comprising a vasoactive prostaglandin selected from the group consisting of prostaglandin E₁ (PGE₁), pharmaceutically acceptable salts thereof, lower alkyl esters thereof and mixtures thereof, a lipophilic component, a penetration enhancer and a shear-thinning polymer thickener. The topical composition is applied to the meatus at the tip of the penis. Typically, the vasoactive prostaglandin, preferably prostaglandin E₁, is present in an amount sufficient to have an effect on the smooth muscle and endothelial cells of the vascular elements of the penis, e.g., an amount generally effective to produce a measurable increase in penile microcirculation, perceptible penile tumescence or penile erection. The composition is preferably administered in repeated doses or sustained release.

In general, the composition includes between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition; a biocompatible polymer; a lipophilic component selected from the group consisting of a C₁ to C₈ aliphatic alcohol, a C₈ to C₃₀ aliphatic ester, a liquid polyol and a mixture thereof; water; and a buffer that provides a buffered pH value for the composition in the range of about 3 to about 7.4. Preferably, the biocompatible polymer is selected from the group consisting of a silastic elastomer, a biodegradable polymer and a shear-thinning polymeric thickener. Preferably, vasoactive prostaglandin is 0.05 to 1 weight percent prostaglandin E₁, based on the total weight of the composition.

In certain preferred embodiments, the penetration enhancer is a shear-thinning polymeric thickener selected from the group consisting of a shear-thinning polysaccharide gum and a shear-thinning polyacrylic acid polymer. When the lipophilic component includes a liquid polyol, the liquid polyol is preferably a polyethylene glycol selected from the group consisting of polyethylene glycol 200, polyethylene glycol 400 and polyethylene glycol 600. In preferred embodiments, the penetration enhancer is selected from the group consisting of an alkyl-(N-substituted amino) alkanoate, an alkyl-2-(N,N-disubstituted amino) alkanoate, an (N-substituted amino) alkanol alkanoate, an (N,N-disubstituted amino) alkanol alkanoate, pharmaceutically acceptable salts thereof and mixtures thereof.

In preferred embodiments, the biocompatible polymer is a biodegradable polymer is selected from the group consisting of a polylactide, a poly(lactide-co-glycolide), a polyorthoester, a polyphosphazene, a polyanhydrides, and a polyphosphoester. In other preferred embodiments, the biodegradable polymer is a biodegradable triblock copolymer selected from the group consisting of a poly(lactide-co-glycolide)-polyethylene glycol-poly(lactide-co-glycolide) copolymer, a polylactide -polyethylene glycol-polylactide copolymer, a polyethylene glycol-poly(lactide-co-glycolide)-polyethylene glycol copolymer and a polyethylene glycol-polylactide-polyethylene glycol copolymer.

In other embodiments, the invention provides a method for restoring microvascular function in a patient which comprises administering to the patient in need of such restoration a vasoactive prostaglandin composition in an amount sufficient to produce a prostaglandin E concentration of about 1 micromolar to about 10 micromolar adjacent to target arterial segments for a time period of at least about four days. In preferred embodiments, the vasoactive prostaglandin composition is applied in the form of a drug depot comprising a vasoactive prostaglandin selected from the group consisting of prostaglandin E₁ (PGE₁), prostaglandin E₂ (PGE₂), pharmaceutically acceptable salts thereof, lower alkyl esters thereof and mixtures thereof, a penetration enhancer and a biocompatible polymer, wherein vascular recovery is demonstrable by objective measures or by clinical findings. Objective measures include microscopic measurements of microvascular outgrowth or laser Doppler flowmetry of penile microcirculation. Vascular recovery can also be demonstrated by clinical findings of penile tumescence or erection. Preferably, the biocompatible polymer is selected from the group consisting of a silastic elastomer, a biodegradable polymer and a shear-thinning polymeric thickener.

In preferred embodiments, the composition comprising the vasoactive prostaglandin and the biocompatible polymer is in fluid communication with the target arterial segment. The vasoactive prostaglandin can be administered continuously or periodically. The arterial segment can be targeted directly by placement of the vasoactive prostaglandin composition adjacent to the internal or external surface of arterial segment in a compartment that is in fluid communication with the target arterial segment. In other embodiments, the arterial segment can be targeted indirectly, by placing the composition in a compartment that is indirectly in fluid communication with the target arterial segment. Without being held to a particular mechanism, it is believed that the treatment of the present invention comprising placing a semisolid prostaglandin composition into the fossa navicularis results in the permeation of prostaglandin E₁ into the tissue of the glans penis and into the corpus spongiosum and the paired corpora cavernosum. The effect of prostaglandin E₁ in the glans produces a prompt increase in blood flow followed by tumescence of the glans and the penis as a whole.

In another embodiment, the invention provides a method for increasing microvascular outgrowth from target arterial segments comprising administering a prostaglandin E₁ composition in an amount sufficient to produce a prostaglandin E₁ concentration in the range of about 10 micromolar to about 30 micromolar adjacent to the target arterial segments for a time period of at least about four days.

In preferred embodiments, the semi-solid vasoactive prostaglandin composition comprises about 0.05 mg to about 0.8 mg of a vasoactive prostaglandin, a penetration enhancer, a shear-thinning polymeric thickener selected from the group consisting of a shear-thinning polysaccharide gum and a shear-thinning polyacrylic acid polymer, a lipophilic component that is selected from the group consisting of a C₁ to C₈ aliphatic alcohol, a C₈ to C₃₀ aliphatic ester, and a mixture thereof; and a buffer system. In a preferred embodiment, the vasoactive prostaglandin is prostaglandin E₁. Preferably the semi-solid composition is packaged in a unit dose and suitably the dose of the prostaglandin E₁ is about 0.05 mg to about 0.8 mg per unit dose, preferably about 0.1 mg to about 0.5 mg per unit dose. In another embodiment, the dose of the prostaglandin E₁ is about 0.1 mg to about 0.3 mg per unit dose.

In preferred embodiments, the penetration enhancer is selected from the group consisting of an alkyl-(N-substituted amino) alkanoate, an alkyl-2-(N,N-disubstituted amino) alkanoate, an (N-substituted amino) alkanol alkanoate, an (N,N-disubstituted amino) alkanol alkanoate, pharmaceutically acceptable salts thereof and mixtures thereof.

The buffer system provides a buffered pH value for the composition in the range of about 3 to about 7.4. A preferred pH value is about 3 to about 6.5, most preferably from about 3.5 to about 6. If desired, stabilizers, preservatives and emulsifiers may be included. In some embodiments, the composition exhibits non-Newtonian rheological properties, suitably comprising a shear-thinning polysaccharide gum or a shear-thinning polyacrylic acid polymer. In one embodiment, the composition is thixotropic. In another embodiment, the composition is pseudoplastic. In a preferred embodiment, the composition has a viscosity of about 5,000 centipoise (cps) to about 20,000 cps, more preferably from about 7,000 cps to about 13,000 cps.

In further embodiments, the present invention provides compositions that are useful for the manufacture of medicaments for the treatment of patients having erectile dysfunction, in particular erectile dysfunction associated with vasculopathy, such as diabetic vasculopathy. Such compositions are also for the manufacture of medicaments for the promoting the recovery of vascular function in a subject having erectile dysfunction, in particular erectile dysfunction associated with vasculopathy, such as diabetic vasculopathy. In other embodiments, the present invention provides compositions that are useful for the manufacture of medicaments for causing microvascular sprouting in a targeted arterial segment.

Other and further aims, purposes, features, advantages, embodiments and the like will be apparent to those skilled in the art from the present specification and the appended claims.

### BRIEF DESCRIPTION OF THE DRAWINGS

FIG. 1 is a graphical representation of the results of a study of microvascular outgrowth produced by contacting in vitro iliac artery segments regions with various concentrations of PGE₁.

### DETAILED DESCRIPTION OF THE INVENTION

Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

“Angiogenesis” means the development of blood vessels.

“Intrameatally” or “meatally” means applying medication to the tip of the penis into the navicularfossa by holding the penis upright, holding the meatus open and dropping the medication into the navicular fossa without introducing the medication container into the meatus.

“Penile tumescence” means the swelling of erectile tissue of the penile, including at least one of the glans, the corpora cavernosa or the corpus spongiosa.

“Alkyl” means the monovalent linear or branched saturated hydrocarbon radical, consisting solely of carbon and hydrogen atoms, having from one to twenty carbon atoms inclusive, unless otherwise indicated. Examples of an alkyl radical include, but are not limited to, methyl, ethyl, propyl, isopropyl, isobutyl, sec-butyl, tert-butyl, pentyl, n-hexyl, octyl, dodecyl, tetradecyl, eicosyl, and the like.

“Lower alkyl” means the monovalent linear or branched saturated hydrocarbon radical, consisting solely of carbon and hydrogen atoms, having from one to six carbon atoms inclusive, unless otherwise indicated. Examples of a lower alkyl radical include, but are not limited to, methyl, ethyl, propyl, isopropyl, tert-butyl, n-butyl, n-hexyl, and the like.

“Lower alkoxy” means the radical —O—R, wherein R is a lower alkyl radical as defined above. Examples of a lower alkoxy radical include, but are not limited to, methoxy, ethoxy, isopropoxy, and the like.

“Halogen” means the radical fluoro, bromo, chloro, and/or iodo.

“Optional” or “optionally” means that the subsequently described event or circumstance may but need not occur, and that the description includes instances where the event or circumstance occurs and instances in which it does not. For example, “optional bond” means that the bond may or may not be present, and that the description includes single, double, or triple bonds.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” of a compound means a salt that is pharmaceutically acceptable, as defined above, and that possesses the desired pharmacological activity of the parent compound. Such salts include:

1. acid addition salts formed with inorganic acids such as hydrochloric acid, hydrobromic acid, hydrofluoric acid, hydroiodic acid, trifluoroacetic acid, sulfuric acid, nitric acid, phosphoric acid, boric acid and the like; or formed with organic acids such as acetic acid, benzenesulfonic acid, benzoic acid, camphorsulfonic acid, p-chlorobenzenesulfonic acid, cinnamic acid, citric acid, cylcopentanepropionic acid, ethanesulfonic acid, 1,2-ethanedisulfonic acid, formic acid, fumaric acid, glucoheptonic acid, gluconic acid, glutamic acid, glycolic acid, hexanoic acid, heptanoic acid, o-(hydroxybenzoyl)benzoic acid, hydroxynaphtoic acid, 2-hydroxyethanesulfonic acid, lactic acid, lauryl sulfuric acid, maleic acid, malic acid, malonic acid, mandelic acid, methanesulfonic acid, 4-methylbicyclo[2.2.2]oct-2-ene-1-carboxylic acid, 4,4′- methylenebis(3-hydroxy-2-ene-1-carboxylic acid), muconic acid, 2-naphthalenesulfonic acid, oxalic acid, 3-phenylpropionic acid, propionic acid, pyruvic acid, salicylic acid, stearic acid, succinic acid, tartaric acid, tertiary butylacetic acid, p-toluenesulfonic acid, trifluoromethanesulfonic acid, trimethylacetic acid, and the like; or

2. salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, methylamine, ethylamine, hydroxyethylamine, propylamine, dimethylamine, diethylamine, trimethylamine, triethylamine, ethylenediamine, hydroethylamine, morpholine, piperazine, and guanidine and the like. Acceptable inorganic bases include aluminum hydroxide, ammonium hydroxide, calcium hydroxide, potassium hydroxide, sodium carbonate, sodium hydroxide and hydrazine. The preferred pharmaceutically acceptable salts are the salts formed from hydrochloric acid, and trifluoroacetic acid.

“Subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

A “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgement of the attending medical or veterinary practitioner, and other factors.

The term “pharmacological effect” as used herein encompasses effects produced in the subject that achieve the intended purpose of a therapy. In one preferred embodiment, a pharmacological effect means that vasospasm symptoms of the subject being treated are prevented, alleviated, or reduced. For example, a pharmacological effect would be one that results in the prevention or reduction of vasospasm in a treated subject.

“Disease state” means any disease, condition, symptom, or indication.

“Treating” or “treatment” of a disease state includes:

1. preventing the disease state, i.e. causing the clinical symptoms of the disease state not to develop in a subject that may be exposed to or predisposed to the disease state, but does not yet experience or display symptoms of the disease state,

2. inhibiting the disease state, i.e., arresting the development of the disease state or its clinical symptoms, or

3. relieving the disease state, i.e., causing temporary or progressive regression of the disease state or its clinical symptoms.

“Pro-drug” means a pharmacologically inactive form of a compound which must be metabolized in vivo by a subject after administration into a pharmacologically active form of the compound in order to produce the desired pharmacological effect. After administration to the subject, the pharmacologically inactive form of the compound is converted in vivo under the influence of biological fluids or enzymes into a pharmacologically active form of the compound. Although metabolism occurs for many compounds primarily in the liver, almost all other tissues and organs, especially the lung, are able to carry out varying degrees of metabolism. Pro-drug forms of compounds may be utilized, for example, to improve bioavailability, mask unpleasant characteristics such as bitter taste, alter solubility for intravenous use, or to provide site-specific delivery of the compound. Reference to a compound herein includes pro-drug forms of a compound.

In a preferred embodiment, the pharmaceutical composition comprises at least one vasoactive prostaglandin, preferably prostaglandin E₁, an alkyl (N-substituted amino) ester, a polymer, a lipophilic component, and an acid buffer system.

Vasoactive prostaglandins are those that act as peripheral vasodilators, including naturally occurring prostaglandins such as PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF3α; semisynthetic or synthetic derivatives of natural prostaglandins, including carboprost tromethamine, dinoprost tromethamine, dinoprostone, lipoprost, gemeprost, metenoprost, sulprostone and tiaprost. Prostaglandin E₁ and prostaglandin E₂ are particularly preferred vasoactive prostaglandins for use in conjunction with the present method.

Additionally, simultaneous administration of one or more non-ecosanoid vasodilators may be desirable and may in some cases exhibit a synergistic effect. The combination of prazosin with prostaglandin E₁ has been found to be particularly advantageous in this regard; the latter drug appears to act as a potentiator for prazosin.

Suitable non-ecosanoid vasodilators include, but are not limited to: nitrates such as nitroglycerin, isosorbide dinitrate, erythrityl tetranitrate, amyl nitrate, sodium nitroprusside, molsidomine, linsidomine chlorhydrate (“SIN-1”) and S-nitroso-N-acetyl-d,1-penicillamine (“SNAP”); amino acids such as L-arginine; long and short acting α-adrenergic blockers such as phenoxybenzamine, dibenamine, phentolamine, tamsulosin and indoramin, especially quinazoline derivatives such as alfuzosin, bunazosin, doxazosin, terazosin, prazosin, and trimazosin; vasodilative natural herbal compositions and bioactive extracts thereof, such as gosyajinki-gan, Satureja obovata, bai-hua qian-hu, lipotab, saiboku-to, vinpocetine, Gingko biloba, bacopa, Gynostemma pentaphyllum, gypenosides, Evodia rutaecarpa, rutaecarpine, dehydroevodiamine, dan-shen, salviae miltiorrhizae radix, shosaikoto, Zizyphi fructus, ginseng and mixtures thereof (U.S. Pat. No. 6,007,824); ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; vasodilators such as nimodepine, pinacidil, cyclandelate, dipyridamole and isoxsuprine; chlorpromazine; haloperidol; yohimbine; trazodone and vasoactive intestinal peptides.

Prostaglandin E₁ is well known to those skilled in the art. Reference may be had to various literature references for its pharmacological activities, side effects, and normal dosage ranges. See for example, Physician's Desk Reference, 51 st Ed. (1997), The Merck Index, 12th Ed., Merck & Co., N.J. (1996), and Martindale The Extra Pharmacopoeia, 28th Ed., London, The Pharmaceutical Press (1982). Prostaglandin E₁ as well as other compounds referenced herein are intended to encompass pharmaceutically acceptable derivatives including physiologically compatible salts and ester derivatives thereof.

The quantity of vasoactive prostaglandin, such as prostaglandin E₁, in the pharmaceutical composition is a therapeutically effective amount and necessarily varies according to the desired dose, the dosage form (e.g., suppository or topical), and the particular form of vasoactive prostaglandin used. The term “prostaglandin” as used generically herein refers to the prostaglandin free acid and pharmaceutically acceptable derivatives thereof, including, for example PGE₁, pharmaceutically acceptable salts and lower alkyl esters thereof (the term “lower alkyl” as used herein means straight chain or branched chain alkyl containing one to four carbon atoms). The composition generally contains

When used in combination with a vasoactive prostaglandin, a piperazinyl quinazoline antihypertensive, such as prazosin, is present in the amount of about 0.1 mg to about 2.0 mg per unit dose, depending on the potency of the particular piperazinyl quinazoline antihypertensive and the type and dose of vasoactive prostaglandin used. The dose and the proportion of vasoactive prostaglandin and the piperazinyl quinazoline antihypertensive can be routinely determined by one of ordinary skill without undo experimentation.

Working alone, most drugs, prostaglandin formulations included, do not sufficiently permeate the skin to provide drug concentration levels comparable to those obtained from other drug delivery routes. To overcome this problem, topical drug formulations typically include a skin penetration enhancer. Skin penetration enhancers also may be referred to as absorption enhancers, accelerants, adjuvants, solubilizers, sorption promoters, etc. Whatever the name, such agents serve to improve drug absorption across the skin. Ideal penetration enhancers not only increase drug flux across the skin, but do so without irritating, sensitizing, or damaging skin. Furthermore, ideal penetration enhancers should not adversely affect the physical qualities of the available dosage forms (e.g. cream or gel), or the cosmetic quality of the topical composition.

A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Büyüktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Suitable penetration enhancers for use in prostaglandin topical compositions are disclosed in U.S. Pat. Nos. 4,980,378, 5,082,866 and 6,118,020 and published International Patent Application WO 95/095590, the contents of all of which are incorporated by reference. Topical compositions employing such penetration enhancers for the delivery of prostaglandins are disclosed in U.S. Pat. Nos. 6,046,244, 6,323,241, 6,414,028, and 6,489,207.

The topical composition of the present invention can contain one or more penetration enhancers. Among the preferred penetration enhancers for the present invention are ethanol, propylene glycol, glycerol, ethyl laurate, isopropyl palmitate, isopropyl myristate, laurocapram (Azone™), dioxolanes (described in U.S. Pat. No. 4,861,764), macrocyclic ketones, HP-101, oxazolidones and biodegradable penetration enhancers (described in U.S. Pat. Nos. 4,980,378 and 5,082,866 to Wong et al. such as alkyl-2-(N,N-disubstituted amino) alkanoates (e.g., dodecyl N,N-dimethylamino isoproprionate (DDAIP)), N,N-disubstituted amino alkanol alkanoates) and mixtures thereof. The penetration enhancer is present in an amount sufficient to enhance the penetration of the vasoactive prostaglandin, e.g., prostaglandin E₁. The specific amount varies necessarily according to the desired release rate and the specific form of prostaglandin E₁ used. Generally, the penetration enhancer is present in an amount ranging from about 0.5 weight percent to about 20 weight percent, based on the total weight of the composition. Preferably, the penetration enhancer is present in an amount ranging from about 1 weight percent to about 10 weight percent of the composition. More preferably, the penetration enhancer is present in an amount ranging from about 1 weight percent to about 5 weight percent of the composition.

In general, suitable penetration enhancers can be chosen from those listed above as well as sulfoxides, alcohols, fatty acids, fatty acid esters, polyols, amides, surfactants, terpenes, alkanones, organic acids and mixtures thereof. See generally Chattaraj, S. C. and Walker, R. B., Penetration Enhancer Classification, pp.5-20 in Maibach, H. I., and Smith, H. E., (eds.), Percutaneous Penetration Enhancers, CRC Press, Inc., Boca Raton, Fla. (1995) and Büyüktimkin, N., et al., Chemical Means of Transdermal Drug Permeation Enhancement, in Gosh, T. K., et al., (eds.) Transdermal and Topical Drug Delivery Systems, Interpharm Press, Inc., Buffalo Grove, Ill. (1997). Suitable sulfoxides include dimethylsulfoxide, decylmethylsulfoxide and mixtures thereof. Suitable alcohols include ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, olcyl alcohol, linolyl alcohol, linolenyl alcohol and mixtures thereof. Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol and mixtures thereof.

Suitable amides include urea, dimethylacetamide, diethyltoluamide, dimethylformamide, dimethyloctamide, dimethyldecamide, 1-alkyl-4-imidazolin-2-one, pyrrolidone derivatives, cyclic amides, hexamethylenelauramide and its derivatives, diethanolamine, triethanolamine and mixtures thereof. Suitable pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-decyl-thioethyl-2-pyrrolidone (HP-101), 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkypyrrolidone, fatty acid esters of N-(2-hydroxymethyl)-2-pyrrolidone and mixtures thereof. Suitable cyclic amides include 1-dodecylazacycloheptane-2-one (laurocapram, Azone®), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexane-2-one, 1-geranylazacyclopentan-2,5-dione, 1-farnesylazacyclopentan-2-one and mixtures thereof.

Suitable surfactants include anionic surfactants, cationic surfactants, nonionic surfactants, bile salts and lecithin. Suitable anionic surfactants include sodium laurate, sodium lauryl sulfate and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxy-poly(oxyethylene)-poly(oxypropyl) poly(oxyethylene)block copolymers, polyoxyethylene ethers, polyoxyethylene sorbitan esters, polyethylene glycol esters of fatty alcohols and mixtures thereof. Suitable α-hydro-ω-hydroxy-poly(oxyethylene)-poly(oxypropyl) poly(oxyethylene)block copolymers include Poloxamers 231, 182, and 184 and mixtures thereof. Suitable polyoxyethylene ethers include 4-lauryl ether (Brij 30), (Brij 93), (Brij 96), 20-oleyl ether (Brij 99) and mixtures thereof. Suitable polyoxyethylene sorbitan esters include the monolaurate (Tween 20, Span 20) the monopalmitate (Tween 40), the monostearate (Tween 60), and the monooleate (Tween 80) and mixtures thereof. Suitable polyethylene glycol esters of fatty acids include the 8-oxyethylene stearate ester (Myrj 45), (Myrj 51), the 40-oxyethylene stearate ester (Myrj 52) and mixtures thereof. Suitable bile salts include sodium cholate, sodium salts of laurocholic, glycolic and desoxycholic acids and mixtures thereof.

Suitable terpenes include D-limonene, α-pinene, β-enrene, α-terpineol, terpinen-4-ol, carvol, carvone, pulegone, piperitone, menthone, menthol, geraniol, cyclohexene oxide, limonene oxide, α-pinene oxide, cyclopentene oxide, 1,8-cineole, ylang ylang oil, anise oil, chenopodium oil, eucalyptus oil and mixtures thereof. Suitable alkanones include N-heptane, N-octane, N-nonane, N-decane, N-undecane, N-dodecane, N-tridecane, N-tetradecane, N-hexadecane and mixtures thereof. Suitable organic acids include citric acid, succinic acid, salicylic acid, salicylates (including the methyl, ethyl and propyl glycol derivatives), tartaric acid and mixtures thereof.

In a preferred embodiment, the penetration enhancer is an alkyl-2-(N-substituted amino)-alkanoate, an (N-substituted amino)-alkanol alkanoate, or a mixture of these. For convenient reference, alkyl-2-(N-substituted amino)-alkanoates and (N-substituted amino)-alkanol alkanoates can be grouped together under the label alkyl (N-substituted amino) esters.

Alkyl-2-(N-substituted amino)-alkanoates suitable for the present invention can be represented as follows:

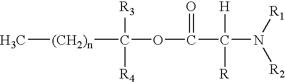

wherein n is an integer having a value in the range of about 4 to about 18; R is a member of the group consisting of hydrogen, C₁ to C₇ alkyl, benzyl and phenyl; R₁ and R₂ are members of the group consisting of hydrogen and C₁ to C₇ alkyl; and R₃ and R₄ are members of the group consisting of hydrogen, methyl and ethyl.

Preferred are alkyl (N,N-disubstituted amino)-alkanoates such as C₄ to C ₁₈ alkyl (N,N-disubstituted amino)-acetates and C₄ to C₁₈ alkyl (N,N-disubstituted amino)-propionates and pharmaceutically acceptable salts and derivatives thereof. Exemplary specific alkyl-2-(N,N-disubstituted amino)-alkanoates include dodecyl 2-(N,N dimethylamino)-propionate (DDAIP);

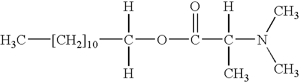

and dodecyl 2-(N,N-dimethylamino)-acetate (DDAA);

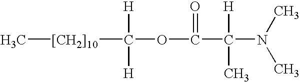

Alkyl-2-(N-substituted amino)-alkanoates are known. For example, dodecyl 2-(N,N-dimethylamino)-propionate (DDAIP) is available from Steroids, Ltd. (Chicago, Ill.). In addition, alkyl-2-(N,N-disubstituted amino)-alkanoates can be synthesized from more readily available compounds as described in U.S. Pat. No. 4,980,378 to Wong et al., which is incorporated herein by reference to the extent that it is not inconsistent. As described therein, alkyl-2-(N,N-disubstituted amino)-alkanoates are readily prepared via a two-step synthesis. In the first step, long chain alkyl chloroacetates are prepared by reaction of the corresponding long chain alkanols with chloromethyl chloroformate or the like in the presence of an appropriate base such as triethylamine, typically in a suitable solvent such as chloroform. The reaction can be depicted as follows:

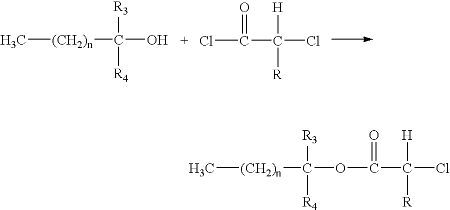

wherein R, R₃, R₄ and n are defined as above. The reaction temperature may be selected from about 10 degrees Celsius to about 200 degrees Celsius or reflux, with room temperature being preferred. The use of a solvent is optional. If a solvent is used, a wide variety of organic solvents may be selected. Choice of a base is likewise not critical. Preferred bases include tertiary amines such as triethylamine, pyridine and the like. Reaction time generally extends from about one hour to three days.

In the second step, the long chain alkyl chloroacetate is condensed with an appropriate amine according to the scheme:

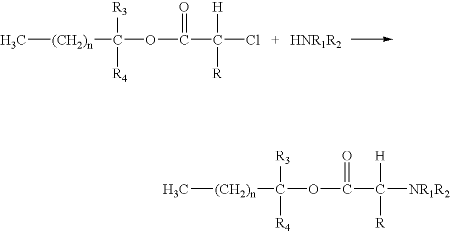

wherein n, R, R₁, R₂, R₃ and R₄ are defined as before. Excess amine reactant is typically used as the base and the reaction is conveniently conducted in a suitable solvent such as ether. This second step is preferably run at room temperature, although temperature may vary. Reaction time usually varies from about one hour to several days. Conventional purification techniques can be applied to ready the resulting ester for use in a pharmaceutical compound.

Suitable (N-substituted amino)-alkanol alkanoates can be represented by the formula:

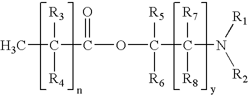

wherein n is an integer having a value in the range of about 5 to about 18; y is an integer having a value in the range of 0 to about 5; and R₁, R₂, R₃, R₄, R₅, R₆, and R₇ are members of the group consisting of hydrogen, C₁ to C₈ alkyl, and C₁ to C₈ aryl; and R₈ is a member of the group consisting of hydrogen, hydroxyl, C₁ to C₈ alkyl, and C₁ to C₈ aryl. The preparation of (N-substituted amino)-alkanol alkanoates and their use as penetration enhancers is disclosed in published PCT International Application WO 95/09590, which is incorporated by reference herein in its entirety.

Preferred are (N-substituted amino)-alkanol alkanoates such as C₅ to C₁₈ carboxylic acid esters and pharmaceutically acceptable salts thereof. Exemplary specific (N,N-disubstituted amino)-alkanol alkanoates include

1-(N,N-dimethylamino)-2-propanol dodecanoate (DAIPD);

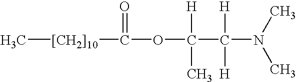

1-(N,N-dimethylamino)-2-propanol myristate (DAIPM);

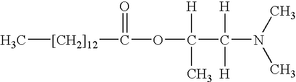

1-(N,N-dimethylamino)-2-propanol oleate (DAIPO);

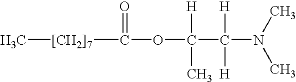

The (N,N-disubstituted amino)-alkanol alkanoates are readily prepared by reacting the corresponding aminoalkinol with lauroyl chloride in the presence of triethylamine. A solvent such as chloroform is optional but preferred. For example, 1-(N,N-dimethylamino)-2-propanol can be reacted with lauroyl chloride in chloroform and in the presence of triethylamine to form 1-(N,N-dimethyl-amino)-2-propanol dodecanoate (DAIPD). Among the suitable penetration enhancers for the present invention DDAIP is generally preferred.

The penetration enhancer is present in an amount sufficient to enhance the penetration of the prostaglandin E₁. The specific amount varies necessarily according to the desired release rate and the specific form of prostaglandin E₁ used. Generally, this amount ranges from about 0.5 percent to about 10 percent, based on the total weight of the composition. In one embodiment, where the vasoactive prostaglandin is prostaglandin E₁, the penetration enhancer is DDAIP in the amount of about 0.01 to about 5 weight percent of the composition.

Additionally, other known transdermal penetration enhancers can also be added, if desired. Illustrative are dimethyl sulfoxide (DMSO), dimethyl acetamide (DMA), 2-pyrrolidone, N,N-diethyl-m-toluamide (DEET), 1-dodecylazacycloheptane-2-one (Azone™, a registered trademark of Nelson Research), N,N-dimethylformamide, N-methyl-2-pyrrolidone, calcium thioglycolate, oxazolidinone, dioxolane derivatives, laurocapram derivatives, and macrocyclic enhancers such as macrocyclic ketones.

Natural and modified polysaccharide gums are also an important ingredient of the composition. Suitable representative gums are those in the natural and modified galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→4). Single membered α-D-manopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carobtree (ceratonia siliqua). Suitable modified polysaccharide gums include ethers of natural or substituted polysaccharide gums, such as carboxymethyl ethers, ethylene glycol ethers and propylene glycol ethers. An exemplary substituted polysaccharide gum is methylcellulose.

Other suitable representative gums include agar gum, carrageenan gum, ghatti gum, karaya gum, rhamsan gum and xanthan gum. The composition of the present invention may contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., N.Y. (1980). Most gums are commercially available in various forms, commonly a powder, and ready for use in foods and topical compositions. For example, locust bean gum in powdered form is available from Tic Gums Inc. (Belcam, Md.).

When present, the polysaccharide gums are present in the range from about 0.1 percent to about 5 percent, based on the total weight of the composition, with the preferred range being from 0.5 percent to 3 percent. In one preferred embodiment, 2.5 percent by weight of a polysaccharide gum is present. Illustrative compositions are given in the examples, below.

An optional alternative to the polysaccharide gum is a polyacrylic acid polymer. A common variety of polyacrylic acid polymer is known generically as “carbomer.” Carbomer is polyacrylic acid polymers lightly cross-linked with polyalkenyl polyether. It is commercially available from the B. F. Goodrich Company (Akron, Ohio) under the designation “CARBOPOL™.” A particularly preferred variety of carbomer is that designated as “CARBOPOL 940.”

Other polyacrylic acid polymers suitable for use are those commercially available under the designations “Pemulen™” (B. F. Goodrich Company) and “POLYCARBOPHIL™” (A. H. Robbins, Richmond, Va.). The Pemulen™ polymers are copolymers of C₁₀ to C₃₀ alkyl acrylates and one or more monomers of acrylic acid, methacrylic acid or one of their simple esters crosslinked with an allyl ether of sucrose or an allyl ether of pentaerythritol. The POLYCARBOPHIL™ enhancer is a polyacrylic acid cross-linked with divinyl glycol. Where polyacrylic acid polymers are present, they represent about 0.5 percent to about 5 percent of the composition, based on its total weight.

The semi-solid composition has a suitably chosen viscosity such that the composition is naturally retained within the fossa navicularis. The semi-solid composition can exhibit Newtonian or non-Newtonian rheological characteristics. In some preferred embodiments, the semi-solid composition of the present invention exhibits non-Newtonian rheological characteristics, i.e. in which the apparent viscosity is dependent on the shear rate applied to the composition. Preferably the composition has “shear-thinning” rheological properties. As used herein, “shear-thinning” refers to a reduction in apparent viscosity (the ratio of shear stress to the shear rate) with increasing shear rate, whether the reduction in apparent viscosity is time independent (pseudoplastic), time dependent (thixotropic) or associated with a yield stress, defined as a stress that must be exceeded before flow starts, (Bingham plastics and generalized Bingham plastics). See, generally, Harris, J., & Wilkinson, W. L., “Non-newtonian Fluid,” pp.856-858 in Parker, S. P., ed., McGraw-Hill Encyclopedia of Physics, Second Edition, McGraw-Hill, New York,1993. A suitable viscosity range of the composition is from about 5,000 centipoise (cps) to about 20,000 cps, preferably from about 7,000 cps to about 13,000 cps.

In certain preferred embodiments, the vasoactive prostaglandin is released over a period of time from a drug depot. While it should be recognized that the release over time of a vasoactive prostaglandin from a semi-solid composition administered meatally and retained within the fossa navicularis is an embodiment of release from a drug depot, in other embodiments, the vasoactive prostaglandin can be released from compositions comprising other polymeric carriers that have been placed in other locations.

In preferred embodiments, a drug depot is formed that comprises a vasoactive prostaglandin and a biocompatible polymer. The biocompatible polymer remains substantially homogenous in the presence of the vasoactive prostaglandin and releases the vasoactive prostaglandin. The biocompatible polymeric material can be hydrophilic or hydrophobic, and can be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, polyolefins, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. The biocompatible polymer may be a protein polymer, fibrin, collagen and derivatives thereof, polysaccharides such as celluloses, starches, dextrans, alginates and derivatives of these polysaccharides, an extracellular matrix component, such as hyaluronic acid, or another biologic agent or a suitable mixture of any of these. The use of an ethylene-vinyl acetate copolymer (EVA, ELVAX-40™, DuPont, Wilmington, Del., USA) and a poly-2-hydroxyethyl-methacrylate polymer (HYDRON™) as drug depots for prostaglandins is known in the art. See, e.g., BenEzra, D., 1978; Form, D. M., & Auerbach, R., 1983; Ziche, M., et al., 1982 and Diaz-Flores, L., et al., Intense vascular sprouting from rat femoral vein induced by prostaglandins E₁ and E₂, Anat Rec., 1994, 238(1):68-76. Such polymers, while biocompatible, have the drawback of requiring removal.

Silicone elastomer drug depots, such as used in Norplant™ (Wyeth) are known in the art. Improvements to drug depots involving modifications of the surface properties of the depot are disclosed in U.S. Pat. No. 6,274,159. Such drug depots, while biocompatible, also have the drawback of requiring removal.

In certain preferred embodiments, the implant is formed from an absorbable or biodegradable polymer. Suitable biodegradable polymers include polylactide (PLA) and poly(lactide-co-glycolide) (PLGA), polyorthoesters, polyphosphazenes, polyanhydrides, and polyphosphoesters. In particularly preferred embodiments, the biodegradable polymer is a polylactide polymer or a poly(lactide-co-glycolide) polymer. Typically the aqueous biodegradable polymer solution is about 9-30% by weight biodegradable copolymer, preferably 20-30% by weight.

The biodegradable polymer comprising the drug depot can be a block copolymer. In certain preferred embodiments the polymer is an ABA- or BAB-type block copolymer, where the A-blocks are a relatively hydrophobic poly(lactide-co-glycolide)(PLGA) or hydrophobic poly(lactide)(PLA) and the B-block is a relatively hydrophilic polyethylene glycol (PEG), having a hydrophobic content of between about 51 to 83% by weight and an overall block copolymer molecular weight of between about 2000 and 4990, that exhibit water solubility at low temperatures and undergo reversible thermal gelation at mammalian physiological body temperatures. The making and use of such block copolymers are disclosed in U.S. Pat. No. 6,117,949 and U.S. Published Patent Application No. 20040001872. The biodegradable triblock polymer is typically used in an aqueous solution of about 9-30% by weight copolymer, preferably 20-30% by weight.

In further preferred embodiments, the prostaglandin drug depot composition is flowable at room temperature and is localized at the deposition site either due to shear-thinning properties or thermal gelation at mammalian physiological body temperatures of the biocompatible polymer.

In preferred embodiments, a solution of a vasoactive prostaglandin in a C₁ to C₈ aliphatic alcohol is added to an aqueous solution of a biodegradable triblock copolymer selected from the group consisting of a PLGA-PEG-PLGA copolymer, a PLA-PEG-PLA copolymer, a PEG-PLGA-PEG copolymer and a PEG-PLA-PEG copolymer to produce a final concentration of 0.001 percent to 1 percent by weight of vasoactive prostaglandin based on the total weight of the composition.

Another important component is a lipophilic component. As used herein “lipophilic component” refers to an agent that is both lipophilic and hydrophilic. One of ordinary skill in the pharmaceutical arts will understand that the lipophilic nature, or “lipophilicity” of a given compound is routinely quantified for comparison to other compounds by using the partition coefficient. The partition coefficient is defined by the International Union of Pure and Applied Chemistry (IUPAC) as the ratio of the distribution of a substance between two phases when the heterogeneous system (of two phases) is in equilibrium; the ratio of concentrations (or, strictly speaking, activities) of the same molecular species in the two phases is constant at constant temperature.

The C₁ to C₈ aliphatic alcohols, the C₂ to C₃₀ aliphatic esters, and their mixtures can serve as lipophilic component. Illustrative suitable alcohols are ethanol, n-propanol and isopropanol, while suitable esters are ethyl acetate, butyl acetate, ethyl laurate, methyl propionate, isopropyl myristate and isopropyl palmitate. As used herein, the term “aliphatic alcohol” includes polyols such as glycerol, propylene glycol and polyethylene glycols. In one embodiment, a mixture of alcohol and ester is preferred, and in particular, a mixture of ethanol and ethyl laurate is preferred.

In some embodiments, the lipophilic component includes at least one liquid polyol. In preferred embodiments, the liquid polyol is a polyethylene glycol selected from the group consisting of polyethylene glycol 200, polyethylene glycol 400 and polyethylene glycol 600. When polyethylene glycol is used, polyethylene glycol is present in the amount of about 1 weight percent to about 25 weight percent, based on the total weight of the composition. A preferred polyethylene glycol is polyethylene glycol 400 (PEG 400). When present, polyethylene glycol 400 is about 1 weight percent to about 25 weight percent, preferably about 3 weight percent to about 20 weight percent, based on the total weight of the composition.

In one embodiment, the C₂ to C₃₀ aliphatic esters, and their mixtures comprising the lipophilic component include C₈ to C₃₀ aliphatic esters of glycerol selected from the group consisting monoglycerides, diglycerides, triglycerides, and mixtures thereof. Suitable aliphatic esters include glyceryl esters of saturated fatty acids, unsaturated fatty acids and mixtures thereof. Suitable saturated fatty acids include caproic acid, caprylic acid, capric acid, lauric acid, myristic acid, palmitic acid, stearic acid, arachidic acid, behenic acid and lignoceric acid. Suitable unsaturated fatty acids include oleic acid, linoleic acid and linolenic acid. Suitable glyceryl esters include glyceryl monooleate, triolein, trimyristin and tristearin, perferably trimyristin.

The concentration of lipophilic component required necessarily varies according to other factors such as the desired semi-solid consistency and the desired skin penetration promoting effects. Suitably the concentration of lipophilic component is in the range of 0.5 percent to 40 percent by weight based on the total weight of the composition. The preferred topical composition contains lipophilic component in the range of 7 percent to 40 percent by weight based on the total weight of the composition.

Where a mixture of aliphatic alcohol and aliphatic ester are employed, the suitable amount of alcohol is in the range of 0.5 percent to 10 percent. In one preferred embodiment, the amount of alcohol is in the range of 5 percent to 15 percent, while that of aliphatic ester is in the range from 2 percent to 15 percent (again based on the total weight of the composition). In another preferred embodiment, the amount of alcohol is in the range of 0.5 percent to 10 percent, while that of aliphatic ester is in the range from 0 percent to 10 percent (again based on the total weight of the composition).

The concentration of lipophilic component required necessarily varies according to other factors such as the desired semi-solid consistency and the desired skin penetration promoting effects. The preferred topical composition contains lipophilic component in the range of 7 percent to 40 percent by weight based on the total weight of the composition. Where a lipophilic component that is a mixture of aliphatic alcohol and aliphatic ester is used, the preferred amount of alcohol is in the range of 5 percent to 15 percent, while that of aliphatic ester is in the range from 2 percent to 15 percent (again based on the total weight of the composition).

An optional, but preferred, component is an emulsifier. Although not a critical factor, a suitable emulsifier generally will exhibit a hydrophilic-lipophilic balance number greater than 10. Sucrose esters, and specifically sucrose stearate, can serve as emulsifiers for the composition. Sucrose stearate is a well-known emulsifier available from various commercial sources. When an emulsifier is used, sucrose stearate present up to about 2 percent, based on the total weight of the composition, is preferred. The preferred amount of sucrose stearate emulsifier can also be expressed as a weight ratio of emulsifier to polysaccharide gum. A ratio of 1 to 6 emulsifier to gum is preferred, and a ratio of 1 to 4 is most preferred to generate the desired semi-solid consistency and separation resistance.

Other emulsifiers are also suitable including polyoxyethylene sorbitan esters, long chain alcohols, preferably cetostearyl alcohol, and fatty acid glycerides. Suitable polyoxyethylene sorbitan esters include the monolaurate (Tween 20, Span 20) the monopalmitate (Tween 40), the monostearate (Tween 60), and the monooleate (Tween 80) and mixtures thereof. Preferred fatty acid glycerides include glyceryl monooleate, triolein, trimyristin and tristearin.

The composition includes an acid buffer system. Acid buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. While there are countless suitable buffers, potassium phosphate monohydrate has proven effective for compositions of the present invention.

The final pH value of the pharmaceutical composition may vary within the physiologically compatible range. Necessarily, the final pH value is not irritating to human skin. Without violating this constraint, the pH may be selected to improve prostaglandin E₁ stability and to adjust consistency when required. In one embodiment, the preferred pH value is about 3.0 to about 7.4, more preferably about 3.0 to about 6.5, most preferably from about 3.5 to about 6.0.

The remaining component of the composition is water, which is necessarily purified. The composition contains water in the range of about 50 to about 90 percent, based on the total weight of the composition. The specific amount of water present is not critical, however, being adjustable to obtain the desired consistency and/or concentration of the other components.

Prostaglandin E₁ stabilizers, coloring agents, rheological agents, and preservatives can be added to the extent that they do not overly limit prostaglandin E₁ skin penetration or prevent the desired semi-solid consistency.

In preferred embodiments, the dosage forms of the semi-solid pharmaceutical composition are creams, gels, ointments, colloidal suspensions and the like, also including but not limited to compositions suitable for use with transdermal patches and like devices.

The ingredients listed above may be combined in any order and manner that produces a stable composition comprising a prostaglandin E₁ evenly dispersed throughout a semi-solid formulation. One available approach to preparing such compositions involves evenly dispersing the polysaccharide gum (or polyacrylic acid polymer) in a premixed water/buffer solution and then thoroughly homogenizing (i.e. mixing) the resulting mixture, which can be labeled “Part A.” When present, the emulsifier is added to the water/buffer solution before dispersing the polysaccharide gum. Any suitable method of adjusting the pH value of Part A to the desired level may be used, for example, by adding concentrated phosphoric acid or sodium hydroxide.

Separately, the prostaglandin E₁ is dissolved with agitation in the lipophilic component, which itself may be a mixture of alcohols, esters, or alcohol with ester. Next, the penetration enhancer is added. Alternatively, when the lipophilic component includes both an alcohol and an ester, the prostaglandin E₁ can be dissolved in the alcohol before adding the penetration enhancer followed by the ester. In either case, the resulting mixture can be labeled “Part B.” The final step involves slow addition (e.g. dropwise) of Part B into Part A under constant mixing.

The resulting topical composition, when compared to exhibits the advantageous properties described above, including improved prostaglandin E₁ permeation and bioavailability without drug overloading, reduced skin damage and related inflammation, and increased flexibility in design of dosage forms. These compositions can be used for prolonged treatment of peripheral vascular disease, male impotency and other disorders treated by prostaglandin E₁, while avoiding the low bioavailability and rapid chemical decomposition associated with other delivery methods. Application of prostaglandin E₁ in a topical composition to the skin of a patient allows a predetermined amount of prostaglandin E₁ to be administered continuously to the patient and avoids undesirable effects present with a single or multiple administrations of larger dosages by injection. By maintaining a sustained dosage rate, the prostaglandin E₁ level in the patient's target tissue can be better maintained within the optimal therapeutic range.

In one embodiment, a composition comprises about 0.01 percent to about 5 percent modified polysaccharide gum; about 0.001 percent to about 1 percent of a vasoactive prostaglandin selected from the group consisting of PGE₁, pharmaceutically acceptable salts thereof, lower alkyl esters thereof and mixtures thereof; about 0.05 percent to about 10 percent DDAIP or salts thereof; about 0.5 percent to about 10 percent of a lower alcohol selected from the group consisting of ethanol, propanol, isopropanol and mixtures thereof; about 0.5 percent to about 10 percent on an ester selected from the group consisting of ethyl laurate, isopropyl myristate, isopropyl laurate and mixtures thereof; based on the weight of the composition, and an acid buffer. Preferably the composition also comprises up to about 2 percent sucrose stearate.

In preferred drug depot embodiments, the vasoactive prostaglandin is 0.05 percent to 1 percent, preferably from 0.1 percent to 0.5 percent prostaglandin E₁, based on the total weight of the composition. Preferably, the biocompatible polymer is selected from the group consisting of a silastic elastomer, a biodegradable polymer and a shear-thinning polymeric thickener. In preferred embodiments, a solution of prostaglandin E₁ in a C₁ to C₈ aliphatic alcohol is added to an aqueous solution of a biodegradable copolymer. Typically the aqueous biodegradable polymer solution is about 9-30% by weight, preferably 20-30% by weight. If necessary, the pH is adjusted to the preferred pH range of about 3.0 to about 7.4, more preferably about 3.0 to about 6.5, most preferably from about 3.5 to about 6.0. If the biodegradable polymer itself does not provide sufficient buffering capacity to maintain the composition in the desired pH range, a suitable buffer, such as a phosphate buffer, may be added as needed. Typically, the composition also includes a lipophilic component selected from the group consisting of a C₁ to C8 aliphatic alcohol, a C₈ to C₃₀ aliphatic ester, and a mixture thereof. In preferred embodiments, the composition includes a penetration enhancer selected from the group consisting of an alkyl-2-(N-substituted amino)-alkanoate ester, an (N-substituted amino)-alkanol-alkanoate, or a mixture thereof.

Optionally the composition also comprises up to about 5 percent emulsifier. Preferably, the composition also comprises up to about 2 percent emulsifier. Suitable emulsifiers include polysorbates such as Tweens, glyceryl monooleate, triolein, trimyristin and tristearin. A preferred emulsifier is trimyristin.

The practice of the present invention is demonstrated in the following examples. These examples are meant to illustrate the invention rather than to limit its scope. Variations in the treating compositions which do not adversely affect the effectiveness of prostaglandin E₁ will be evident to one skilled in the art, and are within the scope of this invention. For example, additional ingredients such as coloring agents, anti-microbial preservatives, emulsifiers, perfumes, prostaglandin E₁ stabilizers, and the like may be included in the compositions as long as the resulting composition retains desirable properties, as described above. When present, preservatives are usually added in amounts of about 0.05 to about 0.30%. Suitable preservatives include methylparabens (methyl PABA), propylparabens (propyl PABA) and butylhydroxy toluene (BHT). Suitable perfumes and fragrances are known in the art; a suitable fragrance is up to about 5 percent myrtenol, preferably about 2 percent myrtenol, based on the total weight of the composition.

The topical composition can further include at least one local anesthetic. Suitable local anesthetics include those approved for topical application (“topical anesthetics”), including, but not limited to ambucaine, amolanone, amylocaine hydrochloride, benoxinate, benzocaine, betoxycaine, biphenamine, bupivacaine, butacaine, butamben, butanilicaine, butethamine, butoxycaine, carticaine, chloroprocaine hydrochloride, cocaethylene, cocaine, cyclomethycaine, dibucaine hydrochloride, dimethocaine, diperodon hydrochloride, dyclonine, ecgonidine, ecgonine, ethyl chloride, etidocaine, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocaine, procaine, propanocaine, proparacaine, propipocaine, propoxycaine hydrochloride, pseudococaine, pyrrocaine, ropivacaine, salicyl alcohol, tetracaine hydrochloride, tolycaine, trimecaine, zolamine and mixtures thereof.

When a topical anesthetic is included, the topical anesthetic comprises about 0.01 to about 10% by weight. Typical topical anesthetics include lidocaine, dyclonine, dibucaine, pharmaceutically acceptable salts and mixtures thereof. In one preferred embodiment, the topical anesthetic is about 0.5 to about 1 percent dyclonine, based on the weight of the composition.

The pharmaceutical preparation is preferably in unit dosage form. In such form the preparation is subdivided into unit doses containing appropriate quantities of the active component. The unit dosage form is a packaged preparation, where the package containing the discrete quantities of the pharmaceutical preparation is, e.g. a rigid plastic dispenser or flexible packet.

Another aspect of the invention is an article of manufacture that comprises a composition for treating erectile dysfunction as described above in a suitable container, preferably in a container such as the dispenser disclosed in U.S. Pat. No. 6,224,573, in combination with labeling instructions. Alternatively, the container can be a tube with a suitable orifice size, such as an extended tip tube, pouch, packet, or squeeze bottle and made of any suitable material, for example rigid plastic or flexible plastic.

The labeling instructions can come in the form of a pamphlet, a label applied to or associated with the packaging of the article of manufacture.

The labeling instructions provide for administering a composition of the invention to the meatus of the penis of a patient suffering from erectile dysfunction, directing the patient to hold the penis upright, hold the meatus open and place the composition in the navicular fossa without introducing the container into the meatus about 5-30 minutes, before sexual intercourse. Printed labeling instructions are functionally related to the composition of the invention inasmuch as such labeling instructions describe a method to treat erectile dysfunction according to the present invention. The labeling instructions are an important aspect of the invention in that before a composition can be approved for any particular use, it must be approved for marketing by the responsible national regulatory agency, such as the United States Food and Drug Administration. Part of that process includes providing a label that will accompany the pharmaceutical composition which is ultimately sold. While the label will include a definition of the composition and such other items such as the clinical pharmacology, mechanism of action, drug resistance, pharmacokinetics, absorption, bioavailability, contraindications and the like, it will also provide the necessary dosage, administration and usage. Thus, the combination of the composition with the dispenser with appropriate treatment instructions is important for the proper usage of the drug once it is marketed to the patient. Such treatment instructions will describe the usage in accordance with the method of treatment set forth herein before.

The fossa navicularis is a natural expanded chamber suitably adapted to receive and retain semisolid medicaments. A semi-solid medicament, such as the composition of the present invention, when placed into the meatus has higher impedance to flow at narrowed exits of this space, the meatus and the urethra. The impedance to flow is proportional to the product of the cross sectional area of the path and the path length. Thus, a semi-solid medication of suitably chosen viscosity is naturally retained within the fossa, facilitating the absorption of active agents such as vasodilators and the like. The viscosity of the composition suitably ranges from about 5,000 cps to about 20,000 cps, preferably from about 7,000 cps to about 13,000 cps. In preferred embodiments, the viscosity of the composition is selected so that about 90% to about 99% of the applied composition is retained in the fossa navicularis for up to about thirty minutes. More preferably about 93% to about 98% of the applied composition, optimally more than 98% is retained in the fossa navicularis for up to about thirty minutes.

The quantity of active component in a unit dose preparation may be varied or adjusted from 0.01 mg to 1 g according to the particular application and the potency of the vasoactive prostaglandin. For example, where the vasoactive prostaglandin is prostaglandin E₁, about 0.05 mg to about 0.8 mg prostaglandin E₁ is present, preferably about 0.1 mg to about 0.5 mg and in another embodiment, about 0.2 mg to about 0.3 mg. The composition can, if desired, also contain other compatible therapeutic agents, such as a piperazinyl quinazoline antihypertensive.

Unless otherwise indicated, each composition is prepared by conventionally admixing the respective indicated components together.

### EXAMPLE 1

Exemplary Compositions

Exemplary Composition A was prepared as follows. Part A was formed by dissolving 0.4 parts prostaglandin E₁ (Alprostadil USP) in 5 parts ethyl alcohol. Next, 5 parts dodecyl 2-(N,N-dimethylamino)-propionate were mixed into the alcohol-prostaglandin E₁ solution, followed by 5 parts ethyl laurate.

Part B was prepared starting from a pH 5.5 water/buffer solution. The water/buffer solution was prepared by adding sufficient potassium phosphate monohydride to purified water to create a 0.1 M solution. The pH of the water/buffer solution was adjusted to 5.5 with a strong base solution (1 N sodium hydroxide) and a strong acid (1 N phosphoric acid). The buffer solution represented about 80 parts of the total composition. All parts specified herein are parts by weight.

To the buffer solution, was added 0.5 parts ethyl laurate. Next, the locust bean gum (in powder form) was dispersed in the buffer solution and homogenized using a homogenizer. Table 1, below, contains a list of ingredients.

The resulting composition was a spreadable, semi-solid suitable for application to the skin without the need for supporting devices such as patches and adhesive strips. The composition was both homogenous in appearance and resistant to separation.

TABLE 1
Topical Prostaglandin E₁ Compositions

| Ingredient | | | | | | | | |
|---|---|---|---|---|---|---|---|---|
| (wt %) | A | B | C | D | E | F | G | H |
| prehydrated | 3 | 3 | 3 | 3 | 3 | 3 | 3 | — |
| locust bean | | | | | | | | |
| gum | | | | | | | | |
| prehydrated | — | — | — | — | — | — | — | 3 |
| modified guar | | | | | | | | |
| gum | | | | | | | | |
| Xanthan gum | — | — | — | — | — | — | — | — |
| water/buffer | 81 | 81 | 81 | 81 | 81 | 81 | 81 | 81 |
| (pH 5.5) | | | | | | | | |
| sucrose stearate | 0.5 | 0.5 | 0.5 | 0.5 | 0.5 | 0.5 | 0.5 | — |
| prostaglandin | 0.1 | 0.2 | 0.3 | 0.4 | 0.4 | 0.5 | 0.4 | 0.3 |
| E₁ | | | | | | | | |
| DDAIP | 5 | 5 | 5 | 5 | 5 | 5 | 5 | 2.5 |
| ethanol | 5 | 5 | 5 | 5 | 5 | 5 | 10 | 5 |
| ethyl laurate | 5 | 5 | 5 | 5 | 5 | 5 | — | 3 |

Additional exemplary compositions B-H are prepared in the same manner using the components listed in Table 1. As noted above, in other embodiments, such as Composition H, the composition may include a modified polysaccharide gum, suitably a modified galactomannan gum, such as a guar gum. Alternatively, a polyacrylic acid polymer may be used instead of the polysaccharide gum.

### EXAMPLE 2

PGE₁ was found to enhance microvascular outgrowth in primary cultures of segments of rat iliac arteries. Each sample was placed on a reduced-growth factor Matrigel coated coverglass slide in a culture dish filled with serum-free medium. PGE₁ (Sigma) was added to the medium at a final concentration of 1, 10, 20, 30, 60, or 100 micromoles (μM). The cultures were incubated at 37° C. in a humidified atmosphere with 5% CO₂. Digital photographs were taken of growth after 96 hours. The control group (6 samples) was incubated in Matrigel in serum free medium without PGE₁.

Growth Factor Reduced Matrigel (Passaniti, A., et al., Lab. Invest. 1992 67:518-528) was purchased from Becton Dickinson (Mountain View, Calif.). Cell culture grade PGE₁ was purchased from Sigma Chemical. (St. Louis, Mo.). RPIM-1640 and other cell culture reagents were purchased from GIBCO Invitrogen Corp. (Grand Island, N.Y.).

Male Sprague-Dawley rats, two-months old, were used in this study. All animal care, treatments, and procedures were approved by the institutional Committee on Animal Research. The rats were sacrificed by intraperitoneal injection of sodium pentobarbital (50 mg/kg) followed by bilateral thoracotomy and the removal of segments of iliac arteries.

The iliac artery segments isolated from Sprague-Dawley rats were sectioned into ringlets and cultured attached to Matrigel-coated glass coverslips. The coverslips were used as the supporting platform to facilitate samples processing for histological staining and examination. Coverslips were coated as follows. Growth Factor Reduced Matrigel (Becton Dickinson, Mountain View, Calif.) was diluted 3-fold in serum-free RPMI-1640 in a 35-mm culture dish on ice. The diluted Matrigel was then spread onto cold sterilized glass coverslips using a sterilized glass slide as spreader. The coated coverslips were placed in 35-mm culture dishes and incubated at 37° C. for 1 hr to allow the Matrigel to solidify. Iliac artery ringlets were placed on top of Matrigel coated coverslips and covered by 50 μl cold Growth Factor Reduced Matrigel which had been kept in liquid form. After a 5-min incubation at 37° C. to allow the Matrigel to polymerize, 3 ml of serum-free RPMI 1640 medium supplemented with 1×penicillin-streptomycin-fangizone (Cell Culture Facility, University of California, San Francisco) was added. PGE₁, (Sigma, Inc., USA) was added to the medium at a final concentration of 1, 10, 20, 30, 60 and 100 μM. The iliac artery ringlet cultures were maintained at 37 ° C. in a humidified atmosphere with 5% CO₂.

After 96 hours of incubation, the iliac artery ringlet cultures were examined. Microvascular growths were photographed using a professional DCS-420 digital camera (Eastman Kodak, Rochester, N.Y.) connected to an Olympus microscope and an Apple Macintosh PowerMac computer. All samples were photographed and the images were stored for later analysis. The digital images were analyzed using ChemiImager 4000 software (Alpha Innotech Corporation, San Leandro, Calif.) to determine the maximum length of the microvascular growths.

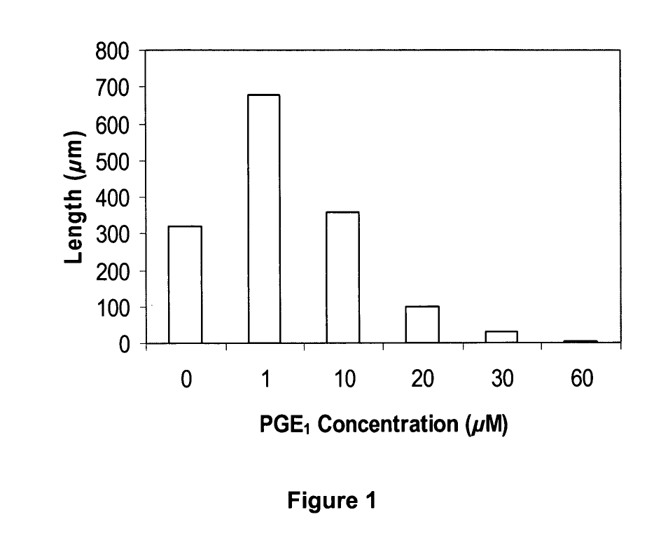

Statistical analysis was performed using computer software from Primer of Biostatistics, 3^{rd} ed. (Glantz SA, McGraw-Hill, Inc. New York, 1992). The data involving different time points were first analyzed by one-way analysis of variance (ANOVA). If ANOVA indicated a significant difference, the Student-Neuman-Keuls test was used to perform pair-wise comparisons. The results are shown in FIG. 1 and Table 2, below.

The relationship between microvascular growth and PGE₁ dose was studied. PGE₁ was added to the medium at a final concentration of 1, 10, 20, 30 or 60 μM. See Table 2, below. The results are shown graphically in FIG. 1. There is a dose-dependent effect, with peak microvascular growth of 680 micrometers at 1 μM PGE₁. Higher doses (10, 20, 30 and 60 μM) produced a smaller maximum microvascular growth.

TABLE 2
PGE₁ Influenced Angiogenesis
(Length of longest microvascular growth, μm)

| PGE₁ Concentration | Length (μm) |
|---|---|
| 0 μM | 320 |
| 1 μM | 680 |
| 10 μM | 360 |
| 20 μM | 100 |
| 30 μM | 30 |
| 60 μM | 5 |

The results are shown graphically in FIG. 1. There is a dose-dependent effect, with peak microvascular growth of 680 micrometers at 1 μM PGE₁. The higher doses of PGE₁ (10, 20, 30 and 60 μM) produced a smaller maximum microvascular growth. The highest doses (20, 30 and 60 μM) apparently reduced the maximum microvascular growth found in the absence of PGE₁.

### EXAMPLE 3

Treatment of Patients Suffering with Erectile Dysfunction Associated with Vasculopathy

A semi-solid prostaglandin topical composition, such as Composition H, is used to promote the recovery of erectile function in a group of patients suffering with erectile dysfunction associated with vasculopathy, such as diabetic vasculopathy. Treatment is performed according to a regime of meatal administration of the prostaglandin topical composition prior to planned sexual intercourse.

Each patient is instructed to place the medication in the navicularfossa by holding the penis upright, holding the meatus open and dropping the medication into the navicular fossa without introducing the medication container into the meatus. Treatment with a prostaglandin composition such as composition H of Example 1 generally produces an erection suitable for vaginal penetration. See U.S. Pat. No. 6,323,241, the contents of which are incorporated herein in their entirety.

In another treatment group, each patient administers a vasoactive prostaglandin dose meatally in a treatment regime that does not rely on plans for sexual intercourse. Preferably a low dose of vasoactive prostaglandin (e.g., 0.2-0.3 mg prostaglandin E₁ per dose) is administered daily via the meatal route for at least four days, more preferably for seven days. Treatment produces an improvement in vascular function that is demonstrable by increased ability to produce an erection suitable for vaginal penetration or by objective measures of penile microcirculation such as laser Doppler flowmetry. See U.S. published patent application 2003/0220292, the contents of which are incorporated herein in their entirety.

In another treatment group, the prostaglandin composition is administered meatally at least once per week, preferably at least three times per week, in a treatment regime lasting at least one month, preferably lasting at least three months. Typically, the prostaglandin E₁ is present in an amount effective produce an increase in penile microcirculation as measured by laser Doppler flowmetry. Increases in penile microcirculation can also be determined clinically by the presence of penile tumescence or penile erection.

While the foregoing is intended to be illustrative of the present invention, the scope is defined by the appended claims. Numerous variations and modifications may be effected without departing from the true spirit and scope of the invention.

We claim:

1. A composition comprising
between 0.001 weight percent and 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition;
a biocompatible polymer;
a lipophilic component selected from the group consisting of a C₁ to C₈ aliphatic alcohol, a C₈ to C₃₀ aliphatic ester, a liquid polyol and a mixture thereof;
water; and
a buffer that provides a buffered pH value for the composition in the range of about 3 to about 7.4.

2. The composition of claim 1 wherein the vasoactive prostaglandin is 0.05 to 1 weight percent prostaglandin E₁, based on the total weight of the composition.

3. The composition of claim 1 wherein the biocompatible polymer is selected from the group consisting of a silastic elastomer, a biodegradable polymer and a shear-thinning polymeric thickener.

4. The composition of claim 3 wherein the biodegradable polymer is selected from the group consisting of a polylactide, a poly(lactide-co-glycolide), a polyorthoester, a polyphosphazene, a polyanhydrides, and a polyphosphoester.

5. The composition of claim 3 wherein the biodegradable polymer is a biodegradable triblock copolymer selected from the group consisting of a poly(lactide-co-glycolide)-polyethylene glycol-poly(lactide-co-glycolide) copolymer, a polylactide-polyethylene glycol-polylactide copolymer, a polyethylene glycol-poly(lactide-co-glycolide)-polyethylene glycol copolymer and a polyethylene glycol-polylactide-polyethylene glycol copolymer.

6. The composition of claim 1 wherein the shear-thinning polymeric thickener selected from the group consisting of a shear-thinning polysaccharide gum and a shear-thinning polyacrylic acid polymer.

7. The composition of claim 1 wherein the liquid polyol is a polyethylene glycol selected from the group consisting of polyethylene glycol 200, polyethylene glycol 400 and polyethylene glycol 600.

8. The composition of claim 1 further comprising a penetration enhancer selected from the group consisting of an alkyl-(N-substituted amino) alkanoate, an alkyl-2-(N,N-disubstituted amino) alkanoate, an (N-substituted amino) alkanol alkanoate, an (N,N-disubstituted amino) alkanol alkanoate, pharmaceutically acceptable salts thereof and mixtures thereof.

9. The composition of claim 1 further comprising an emulsifier.

10. The composition of claim 1 further comprising a fragrance.

11. The composition of claim 1 further comprising a topical anesthetic.

12. A method of promoting the recovery of vascular function in a subject having erectile dysfunction comprising the step of administering a composition comprising a vasoactive prostaglandin selected from the group consisting of prostaglandin E₁, prostaglandin E₂, pharmaceutically acceptable salts thereof, lower alkyl esters thereof, mixtures thereof, a biocompatible polymer and a buffer that provides a buffered pH value for the composition of about 3 to about 7.4, wherein vascular recovery is demonstrable by objective measures or by clinical findings.

13. The method of claim 12 wherein the biocompatible polymer is selected from the group consisting of a silastic elastomer, a biodegradable polymer and a shear-thinning polymeric thickener.

14. The method of claim 12 wherein the vasoactive prostaglandin is between 0.001 weight percent and 1 weight percent of the total weight of the composition.

15. The method of claim 12 wherein the composition further comprises a penetration enhancer is selected from the group consisting of an alkyl-(N-substituted amino) alkanoate, an alkyl-2-(N,N-disubstituted amino) alkanoate, an (N-substituted amino) alkanol alkanoate, an (N,N-disubstituted amino) alkanol alkanoate, pharmaceutically acceptable salts thereof and mixtures thereof.

16. The method of claim 12 wherein the composition further comprises a lipophilic component that is selected from the group consisting of an aliphatic C₁ to C₈ alcohol, an aliphatic C₈ to C₃₀ ester, and a mixture thereof; and water.

17. The method of claim 12 wherein vascular recovery is demonstrable by objective measures of microvascular outgrowth or penile microcirculation.

18. The method of claim 12 wherein vascular recovery is demonstrable by clinical findings of penile tumescence or erection.

19. A method of causing microvascular sprouting in a targeted arterial segment comprising contacting the targeted segment with a solution comprising about 1 micromolar to about 100 micromolar of a vasoactive prostaglandin selected from the group consisting of prostaglandin E₁ and prostaglandin E₂.

20. The method of claim 19 wherein the solution comprises about 1 micromolar to about 60 micromolar prostaglandin E₁.

21. The method of claim 19 wherein the solution comprises about 1 micromolar to about 30 micromolar prostaglandin E₁.

22. The method of claim 19 wherein the solution in contact with the targeted arterial segment is in fluid communication with a composition comprising 0.001 weight percent to 1 weight percent of a vasoactive prostaglandin selected from the group consisting of prostaglandin E₁, prostaglandin E₂, a pharmaceutically acceptable salt thereof, a lower alkyl ester thereof and mixtures thereof, based on the total weight of the composition and a biocompatible polymer selected from the group consisting of biocompatible polymer is selected from the group consisting of a silastic elastomer, a biodegradable polymer and a shear-thinning polymeric thickener.

23. The method of claim 22 wherein the biodegradable polymer is selected from the group consisting of a polylactide, a poly(lactide-co-glycolide), a polyorthoester, a polyphosphazene, a polyanhydrides, and a polyphosphoester.

24. The method of claim 22 wherein the biodegradable polymer is a biodegradable triblock copolymer selected from the group consisting of a poly(lactide-co-glycolide)-polyethylene glycol-poly(lactide-co-glycolide) copolymer, a polylactide-polyethylene glycol-polylactide copolymer, a polyethylene glycol-poly(lactide-co-glycolide)-polyethylene glycol copolymer and a polyethylene glycol-polylactide-polyethylene glycol copolymer.

25. The method of claim 22 wherein the shear-thinning polymeric thickener is selected from the group consisting of a shear-thinning polysaccharide gum and a shear-thinning polyacrylic acid polymer.

26. The method of claim 19 wherein the targeted arterial segment is a segment of a helicine artery, a cavernosal artery, a dorsal penile artery, an internal pudendal artery or an iliac artery.
